(19) United States
(12) Patent Application Publication
(10) Pub. No.: US 2008/0021016 A1
(43) Pub. Date: Jan. 24, 2008

(54) Combined Pharmaceutical Composition for the Inhibition of the Decline of Cognitive Functions

(76) Inventors: Gyorgy Levay, Budakeszi (HU); Istvan Gacsalyi, Budapest (HU); Laszlo Gabor Harsing, Budapest (HU); Gyula Simig, Budapest (HU)

Correspondence Address: K.F. ROSS P.C., 5683 RIVERDALE AVENUE, SUITE 203 BOX 900, BRONX, NY, 10471-0900, US

(21) Appl. No.: 10/592,461

(22) PCT Filed: Mar. 12, 2004
(86) PCT No.: PCT/HU04/00022

§ 371 Date: Jul. 12, 2007

(51) Int. Cl. A61K 31/135; A61K 31/13; A61K 31/4015; A61K 31/437; A61K 31/55; A61P 25/28

(52) U.S. Cl. 514/215; 514/283; 514/423; 514/424; 514/657; 514/659

(57) ABSTRACT

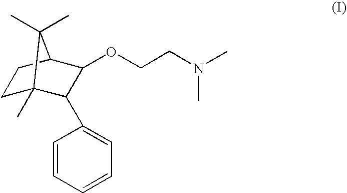

The invention relates to a combined pharmaceutical composition for the inhibition of the decline of cognitive functions comprising as A) component (1R,2S,4R)-(-)-2-[N,N-(dimethylaminoethoxy)]-2-phenyl-1,7,7-trimethylbicyclo]-2-phenyl-1.7.-trimethylbicyclo[2.2.1]heptane of the formula (I) or a pharmaceutically acceptable acid addition salt thereof and as B) component a nootropic, an inhibitor of the acetylcholinesterase enzyme and/or a further pharmaceutical active ingredient which exhibits a beneficial effect on the cognitive processes in admixture with suitable inert pharmaceutical carriers and/or auxiliary agents. The combined pharmaceutical composition according to the present invention can be particularly used for the treatment of Alzheimer disease or other diseases showing similar symptoms, diseases accompanied by malfunctions of intellectual abilities (e.g. mental decline in schizophrenia), mental decline in elderly (dementias in elderly), Korsakoff syndrome, Huntington syndrome, Parkinson syndrome or mental decline produced by alcoholism.

### FIELD OF THE INVENTION

The invention relates to a combined pharmaceutical composition for the inhibition of the decline of cognitive functions.

### TECHNICAL BACKGROUND

(1R,2S,4R)-(-)-2-[N,N-(dimethylaminoethoxy)]-2-phenyl-1,7,7-trimethylbicyclo[2.2.1]heptane of the Formula

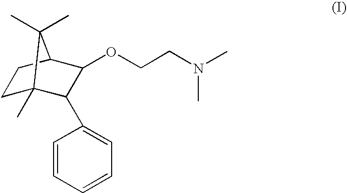

(International Non-Proprietory Name: deramciclane) is an anxiolytic pharmaceutical active ingredient which falls under the general Formula of HU 179,174. The preparation of deramciclane is described inl HU 212,574.

Deramciclane showed considerable effects in different animal models of anxiety and stress. In the Vogel punished drinking test deramciclane was active in 1 and 10 mg/kg after oral administration [Gacsályi et. al, Receptor binding profile and anxiolytic activity of deramciclane (EGIS-3886) in animal models, Drug Dev. Res. 40: p. 338-348, (1997)]. In the social interaction model the compound increased the time spent with social interactions after the single 0.7 mg/kg oral treatment. In the light-dark model [Crawley, J. N. Neuropharmacological specifity of a simple model of anxiety for the behavioural actions of benzodiazepine, Pharmacol. Biochem. Behavior, 15. p. 695-699 (1981)], deramciclane proved to be active in a single oral dose of 3 mg/kg sc. In the marble burying model [Broekkamp, C. L. et al, Major Tranquillizers Can Be Distinguished from Minor Tranquillisers on the Basis of Effects on Marble Burying and Swim-Induced Grooming in Mice. Eur. J. Pharmacol. 126: p. 223-229, (1986)] the molecule was active in 10 and 30 mg/kg after oral treatment.

Regarding the mechanism of action, the compound significantly bound to central 5-HT_{2C and} 5-HT_{2A} receptors [Gacsályi et. al, Receptor binding profile and anxiolytic activity of deramciclane (EGIS-3886) in animal models, Drug Dev. Res. 40. p. 338-348, (1997)].

Numerous clinical studies and observations support that diseases characterised by decline of intellectual and mental functions and/or senile dementia of the elderly are mainly accompanied by abnormality and disability of emotional sphere and mood.

The changes in cognitive functions affecting higher nervous system activity results in disability of adaptation which lead to anxiety and/or depression.

According to the literature, anxiety is present and accelerate the cognitive decline in 68-71% of the patients suffering from Alzheimer disease [Ferretti et al., Anxiety and Alzheimer's disease. J. Geriatr. Psychiatry. Neurol., Spring, 14(1), 52-58 (2001)].

In patients suffering from Huntington disease, a high number of neuropsychiatric symptoms occurred among which anxiety and dysphoria were the most prominent [Paulsen et al., Neuropsychiatric aspects of Huntington's disease. J Neurol. Neurosurg. Psychiatry., 71(3), 310-314, (2001)].

In the dementias of different origin, anxiety are treated by adjuvant pharnacotherapy [Rojas-Fernandez et al., Pharmacotherapy of behavioural and psychological symptoms of dementia. Pharmacotherapy, 21(1) 74-102, (2001)].

### SUMMARY OF THE INVENTION

According to the present invention there is provided a combined pharmaceutical composition for the inhibition of the decline of cognitive functions comprising as A) component (1R,2S,4R)-(-)-2- [N,N-(dimethylaminoethoxy)]-2-phenyl- 1,7,7-trimethylbicyclo[2.2.1]heptane of the Formula I or a pharmaceutically acceptable acid addition salt thereof and as B) component a nootropic, an inhibitor of the acetyl-cholinesterase enzyme and/or a further pharmaceutical active ingredient which exhibits a beneficial effect on the cognitive processes, in admixture with suitable inert pharmaceutical carriers and/or auxiliary agents.

### DETAILED DESCRIPTION OF THE INVENTION

The advantage of the combined pharmaceutical composition of the present invention is that it considerably increases the quality of life of the treated patients by possessing beneficial effect on the cognitive functions (memory, attention, perception, learning) and having at the same time favourable influence on the emotional sphere and mood. The further benefit of the combined pharmaceutical composition of the present invention is that the treated patients are generally aged persons for whom to take several type of medicines is problematic. This could be solved with the help of the combined pharmaceutical composition of the present invention wherein one single medicine is appropriate to handle their conditions resulting in better compliance of the patients.

The present invention is based on the recognition that the anxiolytic, antistress and fear reducing effects of deramciclane of the Formula I or the suitable acid addition salts thereof applied as component A) and the effects of nootropics, inhibitors of acetyl cholinesterase enzyme, or other medicines having beneficial effect on cognitive processes applied as component B) mutually potentiate each other's effect.

The combined pharmaceutical composition of the present invention can be applied to the following indications: Alzheimer disease or diseases showing similar symptoms to Alzheimer disease, diseases accompanied by malfunctions of intellectual abilities (e.g. mental decline in schizophrenia), mental decline in elderly (dementias in elderly), Korsakoff syndrome, Huntington syndrome, Parkinson syndrome or mental decline produced by alcoholism.

The combined pharmaceutical composition according to the present invention comprises as component A) preferably (1R,2S,4R)-(-)-2-[N,N-(dimethylaminoethoxy)]-2-phenyl- 1,7,7-trimethylbicyclo [2.2.1]heptane-2-(E)-butenedioate (1:1).

The combined pharmaceutical composition according to the present invention comprises as component A) particularly preferably (1R,2S,4R)-(-)-2-[N,N-(dimethylaminoethoxy)]-2-phenyl-1,7,7-trimethylbicyclo[2.2.1]heptane or a pharmaceutically acceptable acid addition salt thereof which contains not more than 0.2% of (1R,3S,4R)-(-)-3-[2-N,N-(dimethylaminoethyl)]-1,7,7-trimethylbicyclo [2.2.1]heptane-2-one of the Formula 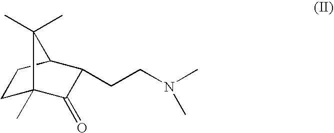
or a pharmaceutically acceptable acid addition salt thereof.

According to a particularly preferable embodiment of the present invention the combined pharmaceutical composition comprises as component A) (1R,2S,4R)-(-)-2-[N,N-(dimethylaminoethoxy)]-2-phenyl-1,7,7-trimethylbicyclo[2.2.1]heptane-2-(E)-butenedioate (1:1) which contains not more than 0.2% of (1R,3S,4R)-(-)-3-[2-N,N-(dimethylaminoethyl)]-1,7,7-trimethylbicyclo[2.2.1 ]heptane-2-one -2-(E)-butenedioate (1:1).

The combined pharmaceutical composition according to the present invention comprises as B) component a nootropic, an inhibitor of the acetyl cholinesterase enzyme and/or a further pharmaceutical active ingredient having beneficial effect on cognitive processes.

As nootropic preferably piracetam, aniracetam, oxiracetam or pramiracetam can be used.

As inhibitor of the acetyl cholinesterase enzyme preferably galantamine, rivastigmin or donezepil can be used.

As B) component furtheron vinpocetin, a calcium antagonist (e.g. nifedipin, nimodipin, amlodipin, felodipin etc.) or an antioxidant (e.g. vitamin E) can be used.

The term “pharmaceutically acceptable acid addition salt” relates to salts formed with pharmaceutically acceptable inorganic or organic acids. For salt formation e.g. hydrochloric acid, hydrogen bromide, sulfuric acid, phosphoric acid, lactic acid, citric acid, tartaric acid, fumaric acid, maleic acid, succinic acid, benzenesulfonic acid, p-toluenesulfonic acid etc. can be used. (1R,2S,4R)-(-)-2-[N,N-(dimethylaminoethoxy)]-2-phenyl-1,7,7-trimethylbicyclo[2.2.1]heptane of the Formula I can be particularly advantageously used in the form of the famarate i.e. as (1R,2S,4R)-(-)-2-[N,N-(dimethylaminoethoxy)]-2-phenyl-1,7,7-trimethylbicyclo[2.2.1]heptane-2-(E)-butenedioate (1:1).

(1R,2S,4R)-(-)-2-[N,N-(dimethylaminoethoxy)]-2-phenyl-1,7,7-trimethylbicyclo[2.2.1]heptane or a pharmaceutically acceptable acid addition salt thereof which contains not more than 0.2% of (1R,3S,4R)-(-)-3-[2-N,N-(dimethylaminoethyl)]-1,7,7-trimethylbicyclo[2.2.1]heptane-2-one of the Formula II or a pharmaceutically acceptable acid addition salt thereof is described in Hungarian patent application HU 1559/99.

The pharmaceutical composition according to the present invention can be prepared in galenic forms generally used in pharmaceutical industry. The compositions may be solid or liquid (e.g. tablets, coated tablets, dragees, capsules, solutions etc.). The pharmaceutical compositions may be administered orally or parenterally, preferably orally. The combined pharmaceutical compositions according to the present invention can be prepared by procedures of pharmaceutical industry known per se.

According to a further aspect of the present invention there is provided a process for the preparation of pharmaceutical compositions for the inhibition of the decline of cognitive functions which comprises admixing as A) component (1R,2S,4R)-(-)-2-[N,N-(dimethylaminoethoxy)]-2-phenyl- 1,7,7-trimethylbicyclo[2.2.1]heptane or a pharmaceutically acceptable acid addition salt thereof and as B) component a nootropic, an inhibitor of the acetyl cholinesterase enzyme and/or a further pharmaceutical active ingredient having beneficial effect on cognitive processes with inert pharmaceutical carriers and/or auxiliary agents and bringing the mixture into a galenic form.

According to a still further aspect of the present invention there is provided the use of a combination comprising as component A) (1R,2S,4R)-(-)-2-[N,N-(dimethylaminoethoxy)]-2-phenyl-1,7,7-trimethylbicyclo[2.2.1]heptane or a pharmaceutically acceptable acid addition salt thereof and as component B) a nootropic, an inhibitor of the acetyl cholinesterase enzyme and/or a further pharmaceutical active ingredient having beneficial effect on cognitive processes for the inhibition of the decline of cognitive functions.

According to a still further aspect of the present invention there is provided the use of a combination comprising as component A) (1R,2S,4R)-(-)-2-[N,N-(dimethylaminoethoxy)]-2-phenyl-1,7,7-trimethylbicyclo[2.2.1]heptane or a pharmaceutically acceptable acid addition salt thereof and as component B) a nootropic, an inhibitor of the acetyl cholinesterase enzyme and/or a further pharmaceutical active ingredient having beneficial effect on cognitive processes for the preparation of a pharmaceutical composition for the inhibition of the decline of cognitive functions.

According to a still further aspect of the present invention there is provided a process for the inhibition of the decline of cognitive functions which comprises administering to the patient in need of such treatment a pharmaceutically effective dose of a combination comprising as component A) (1R,2S,4R)-(-)-2-[N,N-(dimethylaminoethoxy)]-2-phenyl-1,7,7-trimethylbicyclo[2.2.1]heptane or a pharmaceutically acceptable acid addition salt thereof and as component B) a nootropic, an inhibitor of the acetyl cholinesterase enzyme and/or a further pharmaceutical active ingredient having beneficial effect on cognitive processes.

According to a still further aspect of the present invention there is provided the use of (1R,2S,4R)-(-)-2-[N,N-(dimethylaminoethoxy)]-2-phenyl-1,7,7-trimethylbicyclo[2.2.1 ]heptane or a pharmaceutically acceptable acid addition salt thereof for the increase of the effect of nootropics, inhibitors of the acetyl cholinesterase enzyme and/or further pharmaceutical active ingredients which exhibit a beneficial effect on cognitive processes.

Further details of the present invention are to be found in the following Examples without limiting the scope of protection to said Examples.

### EXAMPLES

### Example 1

### Combination of Deramciclane and Galantamine

A preferred dose range is 0.1-50 mg/die of deramciclane and 8-32 mg/die of galantamine. A more preferable dose range is 1-30 mg/die of deramciclane and 10-25 mg/die of galantamine. The most preferred dose range is 2-10 mg/die of deramciclane and 10-20 mg/die of galantamine.

### Example 2

### Combination of Deramciclane and Piracetam

A preferred dose range is 0.1-50 mg/die of deramciclane and 100-1500 mg/die of piracetam. A more preferable dose range is 1-30 mg/die of deramciclane and 500-1200 mg/die of piracetam. The most preferred dose range is 2-10 mg/die of deramciclane and 750-1000 mg/die of piracetam.

### Example 3

### Combination of Deramciclane and Donezepil

A preferred dose range is 0.1-50 mg/die of deramciclane and 0.5-10 mg/die of donezepil. A more preferable dose range is 1-30 mg/die of deramciclane and 1-10 mg/die of donezepil. The most preferred dose range is 2-10 mg/die of deramciclane and 5-10 mg/die of donezepil.

### Example 4

### Combination of Deramciclane and Vinpocetin

A preferred dose range is 0.1-50 mg/die of deramciclane and 1-50 mg/die of vinpocetin. A more preferable dose range is 1-30 mg/die of deramciclane and 5-40 mg/die of vinpocetin. The most preferred dose range is 2-10 mg/die of deramciclane and 10-30 mg/die of vinpocetin.

### Example 5

### Combination of Deramciclane and Vitamin E (Antioxidant)

A preferred dose range is 0.1-50 mg/die of deramciclane and 1-1300 mg/die of vitamin E. A more preferable dose range is 1-30 mg/die of derameiclane and 50-300 mg/die of vitamin E. The most preferred dose range is 2-10 mg/die of deramciclane and 100-300 mg/die of vitamin E.

What we claim is:

1. Combined pharmaceutical composition for the inhibition of the decline of cognitive functions comprising as A) component (1R,2S,4R)-(-)-2-[N,N-(dimethylaminoethoxy)]-2-phenyl-1,7,7-trimethylbicyclo [2.2.1]heptane of the Formula 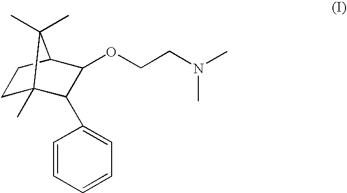
or a pharmaceutically acceptable acid addition salt thereof and as B) component a nootropic, an inhibitor of the acetyl-cholinesterase enzyme and/or a further pharmaceutical active ingredient which exhibits a beneficial effect on the cognitive processes in admixture with suitable inert pharmaceutical carriers and/or auxiliary agents.

2. Combined pharmaceutical composition according to claim 1 for the treatment of Alzheimer disease or other diseases showing similar symptoms, diseases accompanied by malfunctions of intellectual abilities (e.g. mental decline in schizophrenia), mental decline in elderly (dementias in elderly), Korsakoff syndrome, Huntington syndrome, Parkinson syndrome or mental decline produced by alcoholism.

3. Combined pharmaceutical composition according to claim 1 or 2 comprising as A) component (1R,2S,4R)-(-)-2-[N,N-(dimethylaminoethoxy)]-2-phenyl-1,7,7-trimethylbicyclo[2.2.1]heptane-2-(E)-butenedioate (1:1).

4. Combined pharmaceutical composition according to claim 1 comprising as A) component (1R,2S,4R)-(-)-2-[N,N-(dimethylaminoethoxy)]-2-phenyl-1,7,7-trimethylbicyclo[2.2.1]heptane or a pharmaceutically acceptable acid addition salt thereof which contains not more than 0.2% of (1R,3S,4R)-(-)-3-[2-N,N-(dimethylaminoethyl)]-1,7,7-trimethylbicyclo[2.2.1]heptane-2-one of the Formula 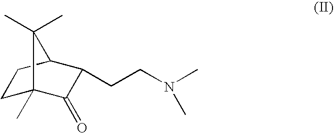
or a pharmaceutically acceptable acid addition salt thereof.

5. Combined pharmaceutical composition according to claim 4 comprising (1R,2S,4R)-(-)-2-[N,N-(dimethylaminoethoxy)]-2-phenyl-1,7,7-trimethylbicyclo[2.2.1]heptane-2-(E)-butenedioate (1:1) which contains not more than 0.2% of (1R,3S,4R)-(-)-3-[2-N,N-(dimethylaminoethyl)]-1,7,7-trimethylbicyclo[2.2.1]heptane-2-one-2-(E)-butenedioate (1:1).

6. Combined pharmaceutical composition according to any of claims 1-5 comprising as B) component piracetam, aniracetam, oxiracetam, pramiracetam, galantamine, rivastigmin, donezepil, vinpocetin, a calcium antagonist or an antioxidant.

7. Process for the preparation of pharmaceutical compositions for the inhibition of the decline of cognitive functions which comprises admixing as A) component (1R,2S,4R)-(-)-2-[N,N-(dimethylaminoethoxy)]-2-phenyl-1,7,7-trimethylbicyclo[2.2.1]heptane or a pharmaceutically acceptable acid addition salt thereof and as B) component a nootropic, an inhibitor of the acetyl cholinesterase enzyme and/or a further pharmaceutical active ingredient having beneficial effect on cognitive processes with inert pharmaceutical carriers and/or auxiliary agents and bringing the mixture into a galenic form.

8. Use of a combination comprising as component A) (1R,2S,4R)-(-)-2-[N,N-(dimethylaminoethoxy)]-2-phenyl-1,7,7-trimethylbicyclo[2.2.1]heptane or a pharmaceutically acceptable acid addition salt thereof and as component B) a nootropic, an inhibitor of the acetyl cholinesterase enzyme and/or a further pharmaceutical active ingredient having beneficial effect on cognitive processes for the inhibition of the decline of cognitive functions.

9. Use of a combination comprising as component A) (1R,2S,4R)-(-)-2-[N,N-(dimethylamninoethoxy)]-2-phenyl-1,7,7-trimethylbicyclo[2.2.1]heptane or a pharmaceutically acceptable acid addition salt thereof and as component B) a nootropic, an inhibitor of the acetyl cholinesterase enzyme and/or a further pharmaceutical active ingredient having beneficial effect on cognitive processes for the preparation of a pharmaceutical composition for the inhibition of the decline of cognitive functions.

10. Process for the inhibition of the decline of cognitive functions which comprises administering to the patient in need of such treatment a pharmaceutically effective dose of a combination comprising as component A) (1R,2S,4R)-(-)-2-[N,N-(dimethylaminoethoxy)]-2-phenyl-1,7,7-trimethylbicyclo[2.2.1]heptane or a pharmaceutically acceptable acid addition salt thereof and as component B) a nootropic, an inhibitor of the acetyl cholinesterase enzyme and/or a further pharmaceutical active ingredient having beneficial effect on cognitive processes.

11. Use of (1R,2S,4R)-(-)-2-[N,N-(dimethylaminoethoxy)]-2-phenyl-1,7,7-trimethylbicyclo[2.2.1]heptane or a pharmaceutically acceptable acid addition salt thereof for the increase of the effect or nootropics, inhibitors of the acetyl cholinesterase enzyme and/or further pharmaceutical active ingredients which exhibit a beneficial effect on cognitive processes.
